(19) United States
(12) Patent Application Publication
(10) Pub. No.: US 2024/0069038 A1
(43) Pub. Date: Feb. 29, 2024

(54) BIOMARKERS COMPOSITION RELATED TO DIAGNOSIS AND PROGNOSIS OF IMMUNOGLOBULIN A NEPHROPATHY AND APPLICATION THEREOF

(71) Applicant: Zhejiang University, Hangzhou (CN)

(72) Inventors: Hong JIANG, Hangzhou (CN); Wenjun ZHAO, Hangzhou (CN); Shi FENG, Hangzhou (CN)

(21) Appl. No.: 17/953,878

(22) Filed: Sep. 27, 2022

(30) Foreign Application Priority Data
Aug. 25, 2022 (CN) 202211025458.3

(51) Int. Cl. G01N 33/68

(52) U.S. Cl. CPC G01N 33/6863; G01N 33/6854; G01N 2800/04

(57) ABSTRACT

Disclosed are a biomarkers composition related to diagnosis and prognosis of immunoglobulin A nephropathy (IgAN) and application thereof, belonging to the field of biomedicine. The biomarkers composition includes at least one of cytokines of FCAR, MBL and MMP-9. It is found after screening and analysis that FCAR, MBL and MMP-9 are associated with the diagnosis of IgAN, especially with severe IgAN. With protein microarray, patients of mild IgAN and severe IgAN, and healthy people are distinguished with high specificity and sensitivity according to the present disclosure.

### CROSS-REFERENCE TO RELATED APPLICATIONS

This application claims priority to Chinese Patent Application No. 202211025458.3, filed on Aug. 25, 2022, the contents of which are hereby incorporated by reference.

### TECHNICAL FIELD

The present disclosure relates to the field of biomedicine, and in particular to a biomarkers composition related to diagnosis and prognosis of Immunoglobulin A nephropathy and application thereof.

### BACKGROUND

Immunoglobulin A nephropathy (IgAN) is a chronic glomerulonephritis caused by abnormal deposition of IgA in the region of glomerulus mesangium. It is the most common primary glomerular disease worldwide and the most serious disease causing end stage renal disease (ESRD) in China. Presently, the pathogenesis of IgAN is not fully elucidated, and therapeutic methods are limited with varying responses to treatment and different prognosis; consequently, early diagnosis of IgAN and timely assessment of the severity of disease progression, together with individualized intervention therapy, are important for effectively delaying the disease and improving the prognosis.

Currently, the diagnosis and assessment of IgAN still requires renal puncture biopsy, which, owing to the invasive procedure, is less acceptable to patients and inconvenient for reproducible operation, as well as timely assessment of disease and treatment efficacy, the application is therefore greatly limited. In recent years, along with the development of biomedical technology, some biomarkers associated with IgAN have been proposed by scholars across the world, and by detecting the biomarkers in terms of expression level in serum and urine alone or in combination, the diagnosis and assessment of IgAN can be facilitated. Nevertheless, further investigation against sensitivity and specificity of these biomarkers during clinical practice is still necessary, as well as in-depth exploration of rather effective biomarkers and biomarkers combinations.

### SUMMARY

In view of solving the problems existing in the prior art, the present disclosure provides a biomarkers composition related to diagnosis and prognosis of immunoglobulin A nephropathy (IgAN) and its application; and by applying the present application, a high-sensitivity and specificity detection of IgAN, especially severe IgAN, is achieved.

To achieve the above objectives, the present disclosure provides the following technical schemes:
a biomarkers composition related to the diagnosis and prognosis of IgAN, where the biomarkers composition includes at least one of cytokines selected from a group of FCAR, MBL and MMP-9.

Optionally, the biomarkers composition includes cytokines of FCAR, MBL and MMP-9.

The present disclosure also provides a product for diagnosing IgAN and/or evaluating the prognosis of IgAN, where the product includes a reagent for detecting the biomarkers composition in terms of expression level.

Optionally, the expression level of the biomarker is calculated according to a following formula: m=0.17*FCAR−4.684*MBL−0.162*MMP-9+8.834; where m represents the expression level of biomarkers composition, with a measure of picogram/milliliter (pg/mL); FCAR, MBL and MMP-9 represent their respective concentrations respectively, with a measure of pg/mL; 8.834 is a constant in the formula calculated by logistic regression model; according to a result of a model, a probability of severe IgAN is calculated as P=e^{m}/1+e^{m}.

Optionally, the product is a protein microarray or a kit.

The present disclosure also provides an application of the reagent for detecting the biomarkers composition in preparing products for assisting diagnosis of IgAN and/or assessing the prognosis of IgAN.

Optionally, the IgAN includes severe IgAN; and the product includes a protein microarray or a kit.

The present application discloses the following technical effects:
it is found through screening and analysis that blood biomarkers FCAR, MBL, MMP-9 in urine are related to the diagnosis of IgAN, especially closely related to the diagnosis of severe IgAN; value of area under curve (AUC) reaches 0.9902 when detecting the FCAR, MBL and MMP-9 in urine in combination, suggesting that combined detection of FCAR, MBL and MMP-9 in urine can be used for diagnosis of IgAN; moreover, the present disclosure also provides a protein microarray for IGAN diagnosis prepared by taking protein microarray as an example, with which the sensitivity and specificity of combined FCAR, MBL and MMP-9 in urine are verified. By combining the above three biomarkers to detect IgAN, the present disclosure achieves noninvasive, convenient and swift operation with good popularization and practical application value.

### BRIEF DESCRIPTION OF THE DRAWINGS

In order to more clearly explain the embodiments of the present disclosure or the technical schemes in the prior art, the following will briefly introduce the drawings that need to be used in the embodiments. Obviously, the drawings in the following description are only some embodiments of the present invention. For those of ordinary skill in the art, other drawings can be obtained according to these drawings without any creative effort.

FIG. 1A, FIG. 1B, FIG. 1C and FIG. 1D illustrate results of differentially expressed protein screening and bioinformatic analysis of serum biomarkers after protein microarray detection; among the drawings, FIG. 1A is a heat map illustrating concentrations and hierarchical cluster of biomarkers in serums of mild Immunoglobulin A nephropathy (IgAN) and normal controls, from where it can be seen that expressions of FGF-23 and MMP-9 are reduced and expressions of other biomarkers are increased in serums of patients of mild IgAN as comparing to that of normal controls; FIG. 1B is a heat map illustrating concentrations and hierarchical cluster of biomarkers in serums of severe IgAN and normal controls, showing that expressions of MBL and MMP-9 are lowered and expressions of other biomarkers are increased in serums of patients of severe IgAN as comparing to that of normal controls; FIG. 1C shows a heat map illustrating concentrations and hierarchical cluster of biomarkers in serums of mild IgAN and severe controls, where expressions of EGF, MBL, MMP-7, and MMP-9 are lowered and expressions of other biomarkers are increased in serums of patients of severe IgAN as comparing to that of patients of mild IgAN; and FIG. 1D shows an intersection of differentially expressed biomarkers (FCAR, MBL, MMP-9) of FIG. 1A and FIG. 1B.

FIG. 2A and FIG. 2B show GO and KEGG analyses of the intersection of the differential expressed biomarkers as described in FIG. 1D.

FIG. 3 shows receiver operating characteristic (ROC) curves of serum FCAR, MBL and MMP-9 for single and combined detection, specifically, A: FCAR; B: MBL; C: MMP-9; D: combination of FCAR, MBL and MMP-9; E: sensitivity detection; and F: specificity detection.

### DETAILED DESCRIPTION OF THE EMBODIMENTS

Now, various exemplary embodiments of the present application will be described in detail. This detailed description should not be taken as a limitation of the present application, but should be understood as a rather detailed description of some aspects, characteristics and embodiments of the present application.

It should be understood that the terms mentioned in the present disclosure are only used to describe specific embodiments, and are not used to limit the present disclosure. In addition, for the numerical range in the present application, it should be understood that each intermediate value between the upper limit and the lower limit of the range is also specifically disclosed. Every smaller range between any stated value or the intermediate value within the stated range and any other stated value or the intermediate value within the stated range is also included in the present disclosure. The upper and lower limits of these smaller ranges can be independently included or excluded from the range.

Unless otherwise stated, all technical and scientific terms used herein have the same meanings commonly understood by those of ordinary skill in the field to which this application relates. Although the present application only describes preferred methods and materials, any methods and materials similar or equivalent to those described herein can be used in the practice or testing of the present disclosure. All documents mentioned in this specification are incorporated by reference to disclose and describe the methods and/or materials related to the documents. In case of conflict with any incorporated documents, the contents of this specification shall prevail.

Without departing from the scope or spirit of the present application, it is obvious to those skilled in the art that many modifications and changes can be made to the specific embodiments of the present specification. Other embodiments obtained from the description of the present application will be obvious to the skilled person. The specification and embodiment of this disclosure are only exemplary.

As used in this paper, the terms “comprising”, “including”, “having” and “containing” are all open terms, meaning including but not limited to.

### Embodiment 1 Screening of Biomarkers Related to Immunoglobulin a Nephropathy (IgAN) Diagnosis

1. Criteria for Biomarkers Screening

(1) the present embodiment adopts literature from PubMed (1966-2020.10), Embase (1966-2020.10), Google Scholar, Cochrane Library databases as objects for screening, and cytokines and proteins that potentially related to the diagnosis and prognosis of IgAN are preliminarily screened; and

20 cytokines and proteins related to the diagnosis and prognosis of IgAN are identified after the above preliminary screening, with results as shown in Table 1:

TABLE 1
Candidate biomarkers

| | | | | | Newcastle- |
|---|---|---|---|---|---|
| | | | | | Ottawa |
| | | | | | Scale |
| | | | | | (NOS) |
| Studies | Biomarkers | Source | Application | Sample size | score |
| Vuong et al., | IgA-Fc fragment | Blood | Prognosis | 109 cases of IgAN | 7 |
| 2010 | receptor (FCAR) | | | and 115 healthy | |
| | | | | controls | |
| Han et al., | Tumor necrosis factor | Blood | Prognosis | 410 cases of IgAN, | 4 |
| 2016 | superfamily | | | 163 healthy controls | |
| | 13(TNFSF13) | | | and 107 diseased | |
| | | | | controls | |
| Lundberg et | Fibroblast growth | Blood | Prognosis | 180 cases of IgAN | 8 |
| al., 2012 | factor 23 (FGF-23) | | | | |
| Meng et al., | Transforming growth | Blood and | Diagnosis and | 100 cases of IgAN, | 6 |
| 2014 | factor β1 (TGF-β1) | glomerulus | prognosis | and 56 healthy | |
| | | | | controls | |
| Taylor et al., | Phosphatidylethanola | Blood and | Diagnosis | 50 cases IgAN, 15 | 5 |
| 2019 | mine binding protein | urine | | healthy controls | |
| | 4 (PEBP-4) | | | | |
| Zwirner et | Activate complement | Blood | Prognosis | 44 cases of IgAN | 4 |
| al., 1997 | C3 (actC3) | | | | |
| Guo et al., | Mannose binding | Blood | Prognosis | 749 cases of IgAN | 8 |
| 2017 | lectin (MBL) | | | and 219 healthy | |
| | | | | controls | |
| Medjeral- | Complement factor H | Blood | Prognosis | 294 cases of IgAN, | 6 |
| Thomas et | related protein 1 | | | 161 healthy controls | |
| al., 2017 | (FHR1) | | | | |
| Zhu et al., | Complement factor H | Blood | Prognosis | 1,126 cases of IgAN, | 5 |
| 2018 | related protein 5 | | | 153 healthy controls | |
| | (FHR5) | | | | |
| Ranieriet | Interleukin-6 (IL- | Urine | Prognosis | 41 cases IgAN, 20 | 7 |
| al., 1996 | 6)/epidermal growth | | | healthy controls | |
| | factor (EGF) ratio | | | | |
| Torres et al., | EGF/monocyte | Urine | Prognosis | 132 cases of IgAN | 8 |
| 2008 | chemoattractant | | | | |
| | protein 1 (MCP-1) | | | | |
| | ratio | | | | |
| Rocchetti et | Aquaporin 2 | Urine | Diagnose | 44 cases of IgAN, 21 | 5 |
| al., 2011 | | | | cases of diseased | |
| | | | | control, and 40 cases | |
| | | | | of healthy control | |
| Liu et al., | C3a, C5a | Urine | Diagnosis | 30 cases of IgAN, 10 | 4 |
| 2014 | | | | diseased controls, and | |
| | | | | 10 healthy controls | |
| Yang et al., | Matrix | Urine | Prognosis | 946 cases of IgAN | 8 |
| 2020 | metalloproteinase 7 | | | | |
| | (MMP-7) | | | | |
| Koideet al., | MMP-9 | Blood | Diagnosis | 45 cases of IgAN, 13 | 8 |
| 1996 | | | | diseased controls, and | |
| | | | | 57 healthy controls | |
| Rocchetti et | Laminin G-like 3 | Urine | Diagnosis | 49 cases of IgAN, 42 | 8 |
| al., 2013 | (LG3) | | | diseased control, and | |
| Zhao et al., | Galactose-deficient | Blood | Prognosis | 40 healthy controls | 8 |
| 2012 | IgA1(Gd-IgA1) | | | 275 cases of IgAN | |
| | | | | and 499 healthy | |
| | | | | controls | |
| Berthoux et | Anti-Gd-IgA1 | Blood | Prognosis | 97 cases of IgAN, 30 | 8 |
| al., 2012 | antibody | | | healthy controls and | |
| | | | | 30 diseased controls | |

(2) A Further Screening is Carried Out Based on the Results as Shown in Table 1 According to Screening Criteria as Follows:
screening criteria: 1) stable and measurable in serum; 2) detectable by protein microarray so as to achieve detection of convenience, rapidness and cost reduction; 3) NOS score reaching 6 or greater than 6;

9 candidate biomarkers with high application value are obtained after the further screening, including FCAR, MBL, MMP-7, MMP-9, IL-6, MCP-1, TGF-β1, EGF, and FGF-23.

### Embodiment 2 Effect Verification of Biomarkers by Using Protein Microarray

1. Sample

Blood samples of 16 patients with mild IgAN and 16 patients with severe IgAN are collected from the Nephrology Center of the First Affiliated Hospital of Zhejiang University, with diagnosis of IgAN confirmed by renal biopsy, where the patients with mild IgAN are confirmed to be of grade I and II by renal biopsy, and the patients with severe IgAN are confirmed to be of or greater that grade III; moreover, blood samples of 16 healthy people are also collected as control, with information of samples as shown in Table 2; then the blood samples are centrifuged to collect upper serums; the above samples are collected with authorization of the patients.

TABLE 2
Clinical sample information

| | Healthy control | Mild IgAN | Severe IgAN | P value |
|---|---|---|---|---|
| Quantity | 16 | 16 | 16 | |
| Gender | Male = 56.25% | Male = 43.75% | Male = 68.75% | 0.362 |
| | Female = 43.75% | Female = 56.25% | Female = 31.25% | |
| Age | 42.2 ± 9.3 | 34.6 ± 10.5 | 39.2 ± 14.3 | 0.184 |

2. Experimental Methods

The 9 biomarkers are detected by QAH-CUST microarray (Quantibody® Custom Arrays, Cat No: QAH-CUST-9, Raybiotech Co., Norcross GA, USA) of RayBio Company in terms of content in the serum and urine;

the samples collected in the previous step (sampled with 2-fold diluted serum, sampled with urine stock solution) are added into the protein microarray and incubated overnight at 4 degree Celsius (° C.), then the microarray is washed and added with detection antibody mixture, followed by incubating at room temperature for 2 hours (h); the microarray is then washed and added with Cy3-conjugated streptavidin, followed by incubating in darkness for 2 h; the microarray is scanned by a InnoScan 300 laser scanner (Innopsys, Parc d'Activites Activestre, 31390 Carbonne-France) after washing, and scanned data are analyzed with a data analysis software of QAH-CUST of Raybiotech Company, including:

(1) Processing of Raw Data and Screening of Expression Difference Markers

Raybiotech software is used to remove the background of the scanned original data and normalize the data between microarrays; in R/Bioconductor, limma data packet is used for moderated t-statistics analysis, and the differential proteins are screened by adjusting p value (or p value corrected by Benjamini-Hochberg (BH) method) and logFC (multiples of expression difference with 2 as a base), where the corrected p value <0.05, as shown in Tables 3-5;

TABLE 3
Concentrations of biomarkers in serums of mild IgAN and normal controls

| | AveExp. | AveExp. | | | | | |
|---|---|---|---|---|---|---|---|
| Marker | Control | Mild IgAN | logFC | P. Value | adj. P. Val | Foldchange | Regulation |
| EGF* | 1.76 | 3.36 | −1.60 | 5.77E−04 | 5.19E−03 | 0.33 | down |
| TGF-β1* | 12.62 | 13.82 | −1.20 | 1.14E−02 | 5.12E−02 | 0.44 | down |
| IL-6* | 1.79 | 2.74 | −0.95 | 2.11E−02 | 6.33E−02 | 0.52 | down |
| MMP-7* | 10.93 | 11.87 | −0.93 | 2.92E−02 | 6.58E−02 | 0.52 | down |
| FGF-23 | 3.87 | 2.28 | 1.59 | 1.38E−01 | 2.48E−01 | 3.01 | up |
| MBL | 10.47 | 10.71 | −0.24 | 5.74E−01 | 6.98E−01 | 0.84 | down |
| MCP-1 | 5.99 | 6.12 | −0.13 | 6.21E−01 | 6.98E−01 | 0.91 | down |
| MMP-9 | 15.29 | 14.95 | 0.34 | 5.03E−01 | 6.98E−01 | 1.27 | up |
| FCAR | 0.28 | 0.48 | −0.20 | 7.63E−01 | 7.63E−01 | 0.87 | down |

*stands for differentially expressed biomarkers;

TABLE 4
Concentrations of biomarkers in serums of severe IgAN and normal controls

| | AveExp. | AveExp. | | | | | |
|---|---|---|---|---|---|---|---|
| Marker | Control | Severe IgAN | logFC | P. Value | adj. P. Val | Foldchange | Regulation |
| EGF* | 1.76 | 3.33 | −1.58 | 6.94E−04 | 3.12E−03 | 0.34 | down |
| MMP-9* | 15.29 | 13.44 | 1.85 | 6.15E−04 | 3.12E−03 | 3.61 | up |
| IL-6* | 1.79 | 3.15 | −1.37 | 1.31E−03 | 3.93E−03 | 0.39 | down |
| FCAR* | 0.28 | 2.35 | −2.07 | 2.93E−03 | 5.28E−03 | 0.24 | down |
| TGF-β1* | 12.62 | 14.06 | −1.43 | 2.81E−03 | 5.28E−03 | 0.37 | down |
| MBL* | 10.47 | 9.35 | 1.12 | 1.23E−02 | 1.85E−02 | 2.18 | up |
| MMP-7 | 10.93 | 11.77 | −0.84 | 5.01E−02 | 6.44E−02 | 0.56 | down |
| MCP-1 | 5.99 | 6.25 | −0.26 | 3.37E−01 | 3.79E−01 | 0.83 | down |
| FGF-23 | 3.87 | 4.32 | −0.45 | 6.71E−01 | 6.71E−01 | 0.73 | down |

*stands for differentially expressed biomarkers;

TABLE 5
Concentrations of biomarkers in serums of mild IgAN and severe IgAN

| | AveExp. | AveExp. | | | | | |
|---|---|---|---|---|---|---|---|
| Marker | Mild IgAN | Severe IgAN | logFC | P. Value | adj. P. Val | Foldchange | Regulation |
| MBL* | 10.71 | 9.35 | 1.37 | 2.67E−03 | 1.98E−02 | 2.58 | up |
| MMP-9* | 14.95 | 13.44 | 1.51 | 4.41E−03 | 1.98E−02 | 2.85 | up |
| FCAR* | 0.48 | 2.35 | −1.87 | 6.77E−03 | 2.03E−02 | 0.27 | down |
| FGF-23 | 2.28 | 4.32 | −2.04 | 5.86E−02 | 1.32E−01 | 0.24 | down |
| IL-6 | 2.74 | 3.15 | −0.41 | 3.10E−01 | 5.57E−01 | 0.75 | down |
| MCP-1 | 6.12 | 6.25 | −0.13 | 6.39E−01 | 8.21E−01 | 0.92 | down |
| TGF-β1 | 13.82 | 14.06 | −0.24 | 6.08E−01 | 8.21E−01 | 0.85 | down |
| MMP-7 | 11.87 | 11.77 | 0.10 | 8.14E−01 | 9.15E−01 | 1.07 | up |
| EGF | 3.36 | 3.33 | 0.03 | 9.52E−01 | 9.52E−01 | 1.02 | up |

*stands for differentially expressed biomarkers;

(2) Clustering Heat Map

function analysis is performed using gplots data packet in R/Bioconductor, where a distance between two samples is calculated as euclidean distance, and a distance between two clusters is calculated as a farthest neighbor method (complete), and a distance between two clusters is defined as a maximum distance of data between two clusters;

(3) Enrichment Analysis of Differential Gene Ontology (GO) and Kyoto Encyclopedia of Genes and Genomes (KEGG) Pathway

with the clusterProfiler package in R/Bioconductor, the fisher's exact test is used for the analysis, with criteria for selection being the number of proteins falling on a term/GO differing by >=2 and p_value <0.05; the term/GO obtained in drawing is arranged in descending order according to the value of Count, and the first 10 results are selected;

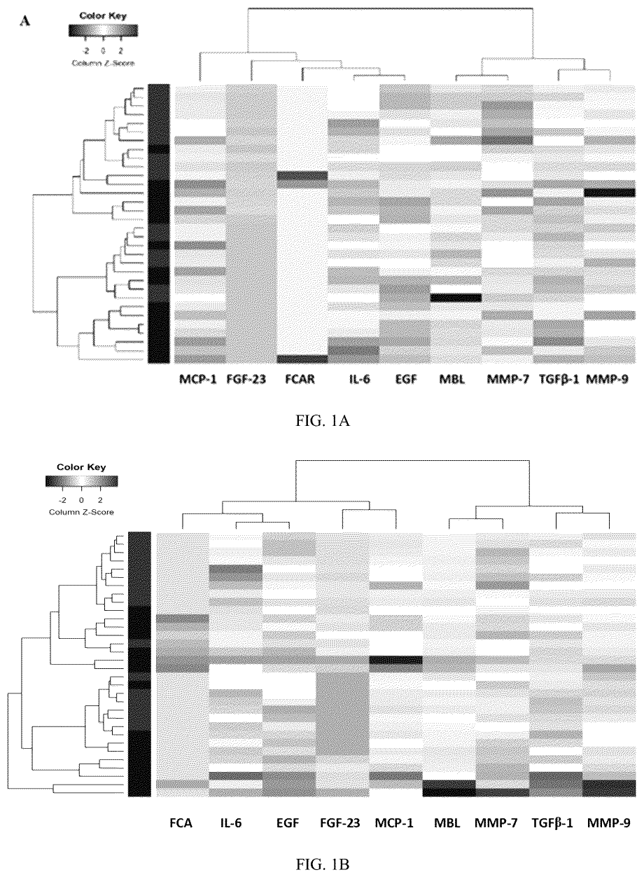

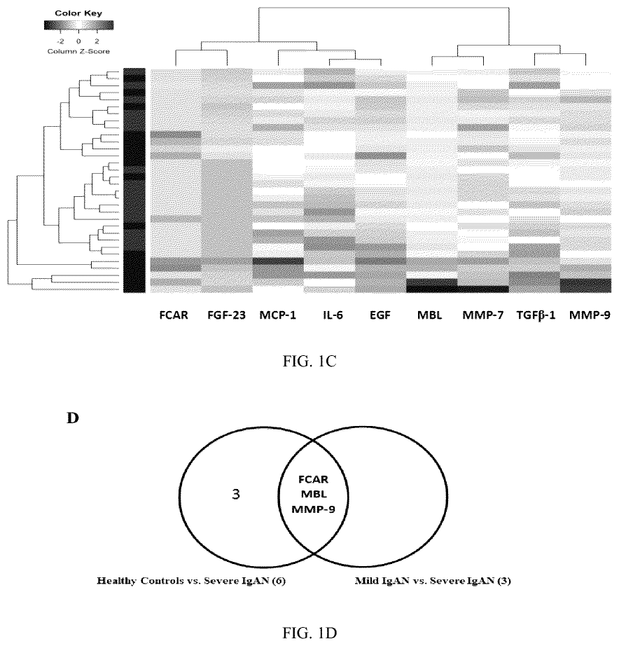

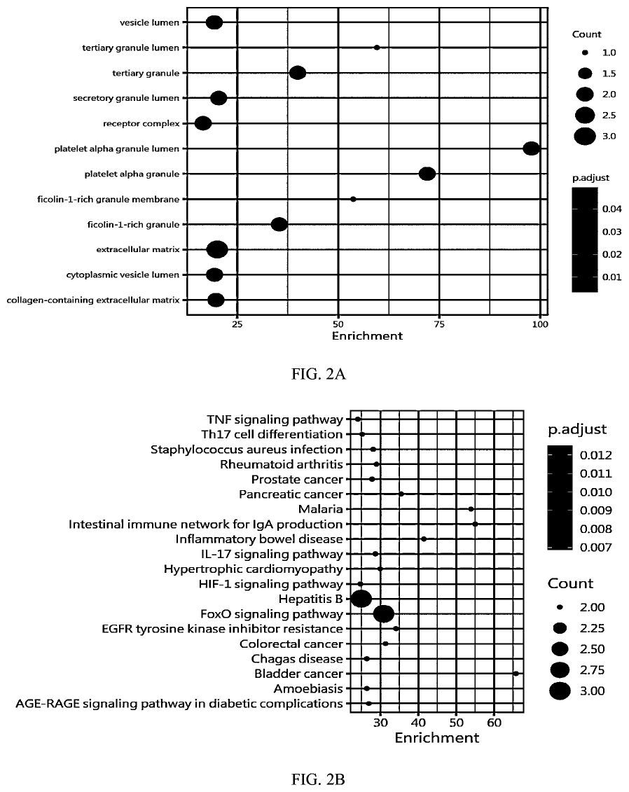

the results of serum biomarkers analysis are shown in FIG. 1A to FIG. 1D, FIG. 2A and FIG. 2B, and intersections of healthy versus severe; intersections of differentially expressed biomarkers between healthy and severe groups, as well as mild versus severe groups, are selected to further screen 3 biomarkers that significantly distinguish healthy controls from IgAN of different degrees, including serum FCAR, MBL and MMP-9.

3. Determination of Diagnostic Value of Biomarkers and their Combinations in IgAN by Receiver Operating Characteristic (ROC) Curve

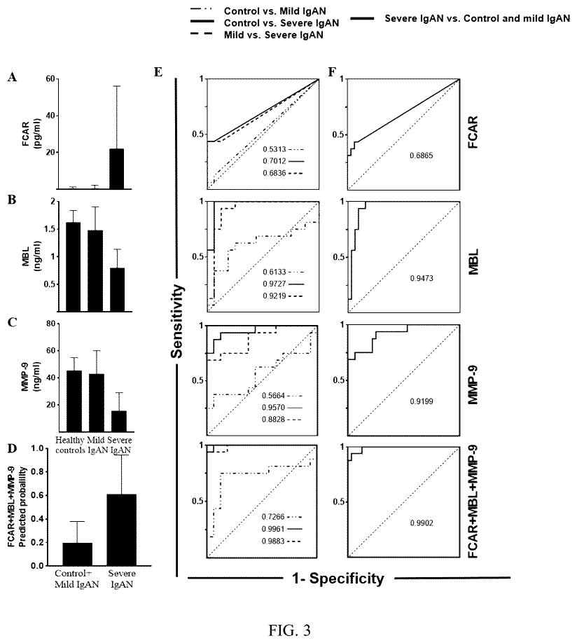

A logistic regression model is build according to concentrations of serum biomarkers measured by protein microarray, and the ROC curves of the above three biomarkers as subjecting to separate detection and combined detection are plotted comprehensively, where the areas under the ROC curves (AUC) are calculated so as to evaluate the diagnostic value, with specific results as shown in FIG. 3.

Results show that the combined detection of blood biomarkers FCAR, MBL and MMP-9 have good sensitivity and specificity for the detection of IgAN, especially severe IgAN; a combined score of these three blood biomarkers is calculated as: 0.17*FCAR concentration (picogram/milliliter, or pg/mL)−4.684*MBL concentration (Nanogram/milliliter, or ng/mL) −0.162*MMP-9 concentration (ng/mL)+8.834; the AUC is calculated to be 0.9902, and when the combined score reaches a boundary value of 0.025, the sensitivity and specificity of detection are 93.75% and 96.88%, respectively, indicating that this biomarker combination has extremely high detection value for detecting severe IgAN; in contrast, the AUC of FCAR, MBL, MMP-9 in blood alone is 0.6865, 0.9473, 0.9199 respectively, which are lower than the score of combined detection of the three biomarkers.

Clinical diagnosis and prognosis of IgAN in prior art are still based on renal biopsy, causing invasive and possible complications as well as limitation in clinical application, where promptness and effectiveness are highly required; IgAN severity can also be assessed by some traditional indicators, such as proteinuria, blood pressure and estimated glomerular filtration rate (eGFR) at biopsy; such indicators are less invasive yet non-specific, and therefore cannot serve to improve individualized treatment as fail to provide potential pathophysiological information. Some new biomarkers discovered in recent years, unsatisfactory specificity and sensitivity, still cannot replace invasive renal biopsy. However, the serum biomarkers combination developed by the present application achieves clinical noninvasive diagnosis of IgAN, especially severe IgAN, and a combined detection method with biomarkers for noninvasive diagnosis of IgAN is also provided after literature review, preliminary statistical analysis, and further verification in clinical samples, which is verified by ROC curve analysis to have good sensitivity and specificity, and can hence be utilized to prepare protein microarray for diagnosis of IgAN; moreover, the combined adaptation of the biomarkers can also be applied in combination to products such as commercial kits.

The above-mentioned embodiments only describe the preferred mode of the present application, but do not limit the scope of the application. On the premise of not departing from the design spirit of the application, all kinds of modifications and improvements made by ordinary technicians in the field to the technical scheme of the application shall fall within the scope of protection determined by the claims of the application.

What is claimed is:

1. A biomarkers composition related to diagnosis and prognosis of immunoglobulin A nephropathy (IgAN), comprising at least one of cytokines selected from a group of FCAR, MBL and MMP-9.

2. The biomarkers composition according to claim 1, wherein the biomarkers composition comprises cytokines of FCAR, MBL, and MMP-9.

3. A product for diagnosing and evaluating prognosis of IgAN, comprising a reagent for detecting the biomarkers composition according to claim 1 in terms of expression level.

4. The product according to claim 3, wherein a score of the biomarkers composition is calculated as follows: m=0.17*FCAR−4.684*MBL−0.162*MMP-9+8.834, where m represents an expression level of biomarkers composition, with a measure of picogram/milliliter (pg/mL); FCAR, MBL and MMP-9 represent respective concentrations, with a measure of pg/mL; and 8.834 is a constant in the formula calculated by a logistic regression model.

5. The product according to claim 3, wherein the product is a protein microarray or a kit.

6. An application of the reagent for detecting the biomarkers composition according to claim 1 in preparing products for assisting diagnosis of IgAN and assessing the prognosis of IgAN.

7. The application according to claim 6, wherein the IgAN comprises severe IgAN, and the product comprises a protein microarray or a kit.
